(19) United States
(12) Patent Application Publication
(10) Pub. No.: US 2014/0187528 A1
(43) Pub. Date: Jul. 3, 2014

(54) TAZOBACTAM ARGININE ANTIBIOTIC COMPOSITIONS

(71) Applicant: Cubist Pharmaceuticals, Inc., Lexington, MA (US)

(72) Inventors: Jan-Ji Lai, Westborough, MA (US); Jian-Qiao Gu, Lexington, MA (US); Pradip M. Pathare, Lexington, MA (US); Valdas Jurkauskas, Cambridge, MA (US); Joseph Terracciano, Concord, MA (US); Nicole Miller Damour, Belmont, MA (US)

(21) Appl. No.: 14/200,383

(22) Filed: Mar. 7, 2014

Related U.S. Application Data
(63) Continuation of application No. PCT/US2013/062256, filed on Sep. 27, 2013.
(60) Provisional application No. 61/706,399, filed on Sep. 27, 2012.

(51) Int. Cl. A61K 31/431; A61K 31/155; A61K 31/546

(52) U.S. Cl. CPC A61K 31/431; A61K 31/546; A61K 31/155
USPC 514/192

(57) ABSTRACT

This disclosure provides compositions comprising a beta-lactam compound and crystalline tazobactam arginine, and related methods and uses of these compositions.

### RELATED APPLICATION

The present application claims priority to U.S. Provisional Application No. 61/706,399, filed Sep. 27, 2012, which is incorporated herein by reference in its entirety.

### TECHNICAL FIELD

This disclosure relates to pharmaceutical compositions comprising tazobactam arginine and related methods and uses thereof.

### BACKGROUND

The cephalosporin (6R,7R)-3-[(5-amino-4-{[(2-aminoethyl)carbamoyl]amino}-1-methyl-1H-pyrazol-2-ium-2-yl)methyl]-7-({(2Z)-2-(5-amino-1,2,4-thiadiazol-3-yl)-2-[(1-carboxy-1-methylethoxy)imino]acetyl}amino)-8-oxo-5-thia-1-azabicyclo[4.2.0]oct-2-ene-2-carboxylate (also referred to as ceftolozane, or (6R,7R)-3-[5-Amino-4-[3-(2-aminoethyl)ureido]-1-methyl-1H-pyrazol-2-ium-2-ylmethyl]-7-[2-(5-amino-1,2,4-thiadiazol-3-yl)-2-[(Z)-1-carboxy-1-methylethoxyimino]acetamido]-3-cephem-4-carboxylic acid) is an antibacterial agent. The antibacterial activity of ceftolozane is believed to result from its interaction with penicillin binding proteins (PBPs) to inhibit the biosynthesis of the bacterial cell wall which acts to stop bacterial replication. Ceftolozane can be combined (e.g., mixed) with a β-lactamase inhibitor (“BLI”), such as tazobactam. Tazobactam is a BLI against Class A and some Class C β-lactamases, with well-established in vitro and in vivo efficacy in combination with active β-lactam antibiotics.

Antibiotic pharmaceutical compositions can include a beta-lactam compound having antibiotic properties (i.e., an antibiotic compound possessing one or more beta-lactam moieties) and a BLI, such as tazobactam. Beta-lactam compounds can be formulated with and/or administered in combination with, beta-lactamase inhibiting compounds (e.g., tazobactam and salts thereof) in order to mitigate the effects of bacterial beta-lactamases. For example, the combination of ceftolozane and tazobactam in a 2:1 weight ratio is an antibiotic pharmaceutical composition (“CXA-201”) formulated for parenteral administration. CXA-201 displays potent antibacterial activity in vitro against common Gram-negative and selected Gram-positive organisms. CXA-201 is a broad-spectrum antibacterial with in vitro activity against Enterobacteriaceae including strains expressing extended spectrum (3-lactamases-resistant (MIC₉₀=1 μg/mL), as well as Pseudomonas aeruginosa (P. aeruginosa) including multi-drug resistant strains (MIC₉₀=2 μg/mL). CXA-201 is a combination antibacterial with activity against many Gram-negative pathogens known to cause intrapulmonary infections, including nosocomial pneumonia caused by P. aeruginosa.

### SUMMARY

Provided herein are compositions comprising beta-lactam compounds (e.g., ceftolozane, or a pharmaceutically acceptable salt thereof) and tazobactam arginine, including pharmaceutical compositions comprising beta-lactam compounds and crystalline tazobactam arginine, and pharmaceutical compositions prepared using beta-lactam compounds and crystalline tazobactam arginine. Methods of making and related uses of these combinations are also provided.

Particularly, pharmaceutical compositions can comprise a beta-lactam compound and crystalline tazobactam arginine. Crystalline compounds of tazobactam arginine can also possess properties that are beneficial to the preparation of various drug formulations and pharmaceutical compositions. Pharmaceutical compositions comprising crystalline forms of tazobactam arginine, or pharmaceutical compositions prepared using crystalline forms of tazobactam arginine, may exhibit beneficial properties including desired levels of chemical stability over time and/or in the presence of heat and humidity, and reduced levels of impurities. Compared with previous crystalline forms of tazobactam, certain crystalline tazobactam arginine solid forms are provided herein that have the advantageous characteristic of being less hygroscopic. These crystalline tazobactam arginine solid forms can have good thermal stability and light stability in the process of preparation, packing, transportation and storage.

Preferably, the beta-lactam compound used in combination with crystalline tazobactam arginine is (6R,7R)-3-[(5-amino-4-{[(2-aminoethyl)carbamoyl]amino}-1-methyl-1 H-pyrazol-2-ium-2-yl)methyl]-7-({(2Z)-2-(5-amino-1,2,4-thiadiazol-3-yl)-2-[(1-carboxy-1-methylethoxy)imino]acetyl}amino)-8-oxo-5-thia-1-azabicyclo[4.2.0]oct-2-ene-2-carboxylate, or a pharmaceutically acceptable isomer, salt, ester, hydrate, solvate, or combination thereof.

In another aspect, provided herein is a method of making a pharmaceutical composition comprising combining crystalline tazobactam arginine and a beta-lactam compound. In one embodiment, the method comprises the steps of: (1) preparing a mixture comprising crystalline tazobactam arginine and a beta-lactam compound; (2) preparing an aqueous solution from the mixture; and (3) lyophilizing the solution to obtain said pharmaceutical composition.

Also provided are pharmaceutical compositions prepared according to the above method.

The above pharmaceutical compositions can be used in methods for the treatment of bacterial infections in a mammal, the methods comprising administering to said mammal a therapeutically effective amount of the pharmaceutical compositions.

### BRIEF DESCRIPTION OF THE DRAWINGS

FIG. 1 depicts the X-ray powder diffraction pattern of polymorph Ia.

FIG. 2 depicts the differential scanning calorimetry (DSC) thermogram of polymorph Ia.

FIG. 3 depicts the thermogravimetric analysis (TGA) curve of polymorph Ia.

FIG. 4 depicts the X-ray powder diffraction pattern of polymorph Ib.

FIG. 5 depicts impurities observed in Example 3.

### DETAILED DESCRIPTION

Pharmaceutical compositions comprising one or more drug substances or excipients can be prepared in a variety of ways, including, for example, blending and lyophilization (also known as “co-lyophilization”). As is known to those skilled in the art, lyophilization is a process of freeze-drying in which water is sublimed from a frozen solution of one or more solutes. Specific methods of lyophilization are described in Remington's Pharmaceutical Sciences, Chapter 84, page 1565, Eighteenth Edition, A. R. Gennaro, (Mack Publishing Co., Easton, Pa., 1990).

The formulation of pharmaceutical compositions can be selected to minimize decomposition of the constituent drug substances and to produce a composition that is stable under a variety of storage conditions. Surprisingly, pharmaceutical compositions comprising crystalline forms of tazobactam arginine (e.g., pharmaceutical compositions prepared using crystalline forms of tazobactam arginine) have been observed to exhibit beneficial properties including desired levels of chemical stability over the course of time and/or in the presence of heat and humidity, and reduced levels of impurities. In a particular embodiment described herein (see Example 4), a pharmaceutical composition prepared from crystalline tazobactam arginine and ceftolozane was observed to undergo less decomposition of both tazobactam and ceftolozane over time.

The beneficial properties of the above pharmaceutical compositions may be attributable to the unique physical properties of crystalline tazobactam arginine. Tazobactam arginine can occur in an amorphous solid form or in a crystalline solid form. Crystalline solid forms of tazobactam arginine can exist in one or more unique polymorph forms, which can additionally comprise one or more equivalents of water or solvent (i.e., hydrates or solvates, respectively).

Tazobactam arginine is the salt of the conjugate base of tazobactam and the conjugate acid of (S)-2-amino-5-guanidinopentanoic acid (L-arginine) in a 1:1 ratio, as represented by the structure below.

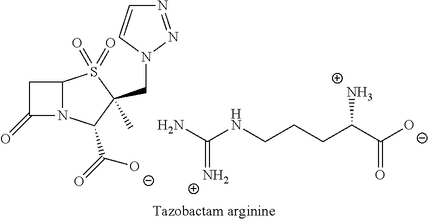 [SMILES: C[C@]1(Cn2ccnn2)[C@H](C(=O)[O-])N2C(=O)CC2S1(=O)=O ; NC(=[NH2+])NCCC[C@H]([NH3+])C(=O)[O-]]

Accordingly, provided herein are compositions comprising a beta-lactam compound and crystalline tazobactam arginine, or hydrates and solvates thereof, particularly crystalline tazobactam arginine polymorph Ia, (also referred to herein as “polymorph Ia” or “tazobactam arginine polymorph Ia”) and crystalline tazobactam arginine polymorph Ib (also referred to herein as “polymorph Ib” or “tazobactam arginine polymorph Ib”).

### Polymorphism

The ability of a substance to exist in more than one crystal form is defined as polymorphism; the different crystal forms of a particular substance are referred to as “polymorphs.” In general, polymorphism is affected by the ability of a molecule of a substance to change its conformation or to form different intermolecular or intra-molecular interactions, particularly hydrogen bonds, which is reflected in different atom arrangements in the crystal lattices of different polymorphs. In contrast, the overall external form of a substance is known as “morphology,” which refers to the external shape of the crystal and the planes present, without reference to the internal structure. Crystals can display different morphology based on different conditions, such as, for example, growth rate, stirring, and the presence of impurities.

The different polymorphs of a substance can possess different energies of the crystal lattice and, thus, in solid state they can show different physical properties such as form, density, melting point, color, stability, solubility, dissolution rate, etc., which can, in turn, affect the stability, dissolution rate and/or bioavailability of a given polymorph and its suitability for use as a pharmaceutical and in pharmaceutical compositions.

Access to different polymorphs of tazobactam arginine is desirable for other reasons as well. One such reason is that different polymorphs of a compound (e.g., tazobactam arginine) can incorporate different impurities, or chemical residues, upon crystallization. Certain polymorphs incorporate very little, or no, chemical residues. Accordingly, the formation of certain polymorph forms of a compound may result in purification of the compound.

Tazobactam arginine polymorph Ia exhibits low hygroscopicity relative to amorphous tazobactam arginine and amorphous tazobactam sodium. Low hygroscopicity of a solid compound is desirable for several reasons. For example, compounds that are highly hygroscopic may be chemically unstable, or unsuitable for formulating as a drug product due to changes of the drug form's physical characteristics (e.g., bulk density, dissolution rate, etc.) that can occur if it is stored in settings with varying relative humidity. Also, hygroscopicity can impact large-scale manufacturing and handling of a compound. For example, it may be difficult to determine the true weight of a hygroscopic active agent when preparing a pharmaceutical composition comprising that agent.

### Characterization of Solid Crystalline Forms of Tazobactam Arginine

In certain embodiments, the compounds used in the combination therapies described herein are identifiable on the basis of characteristic peaks in an X-ray powder diffraction analysis. X-ray powder diffraction, also referred to as XRPD, is a scientific technique using X-ray, neutron, or electron diffraction on powder, microcrystalline, or other solid materials for structural characterization of the materials.

As used herein, the phrase “degrees 2-Theta±0.3°” indicates that each subsequently listed angle has an error of ±0.3°; the phrase “degrees 2-Theta±0.2°” indicates that each subsequently listed angle has an error of ±0.2°; and the phrase “degrees 2-Theta±0.1°” indicates that each subsequently listed angle has an error of ±0.1°. For example, the phrase “degrees 2-Theta±0.2° at angles of 1, 2 and 3” is equivalent to the phrase “degrees 2-Theta at angles of 1±0.2°, 2±0.2° and 3±0.2°”.

One embodiment of crystalline tazobactam arginine used in the combination therapies described herein is referred to as polymorph Ia (also referred to herein as “tazobactam arginine polymorph Ia”) and is characterized by an X-ray powder diffraction pattern having one or more characteristic peaks expressed in degrees 2-Theta at angles selected from about 8.9°±0.3°, about 18.0°±0.3° and about 21.2°±0.3°. In another embodiment, polymorph Ia is characterized by an X-ray powder diffraction pattern having one or more peaks expressed in degrees 2-Theta at angles selected from about 4.8°±0.3°, about 11.3°±0.3° and about 14.9°±0.3°. In still another embodiment, polymorph Ia is characterized by an X-ray powder diffraction pattern having one or more peaks expressed in degrees 2-Theta at angles selected from about 19.4°±0.3°, about 22.8°±0.3° and about 24.3°±0.3°.

In another embodiment, polymorph Ia is characterized by an X-ray powder diffraction pattern having 3-6 peaks expressed in degrees 2-Theta at angles selected from about 8.9°±0.3°, about 18.0°±0.3°, about 21.2°±0.3°, about 4.8°±0.3°, about 11.3°±0.3°, about 14.9°±0.3°, about 19.4°±0.3°, about 22.8°±0.3° and about 24.3°±0.3°. In a particular embodiment, polymorph Ia is characterized by an X-ray powder diffraction pattern having characteristic peaks expressed in degrees 2-Theta at angles of about 8.9°±0.3°, about 18.0°±0.3° and about 21.2°±0.3°.

In another embodiment, polymorph Ia is characterized by an X-ray powder diffraction pattern having 3-6 peaks expressed in degrees 2-Theta at angles selected from about 8.9°±0.3°, about 18.0°±0.2°, about 21.2°±0.2°, about 4.8°±0.2°, about 11.3°±0.2°, about 14.9°±0.2°, about 19.4°±0.2°, about 22.8°±0.2° and about 24.3°±0.2°. In a particular embodiment, polymorph Ia is characterized by an X-ray powder diffraction pattern having characteristic peaks expressed in degrees 2-Theta at angles of about 8.9°±0.2°, about 18.0°±0.2° and about 21.2°±0.2°.

In yet another embodiment, polymorph Ia is characterized by an X-ray powder diffraction pattern having 6-9 peaks expressed in degrees 2-Theta at angles selected from about 8.9°±0.3°, about 18.0°±0.3°, about 21.2°±0.3°, about 4.8°±0.3°, about 11.3°±0.3°, about 14.9°±0.3°, about 19.4°±0.3°, about 22.8°±0.3° and about 24.3°±0.3°. In a particular embodiment, polymorph Ia is characterized by an X-ray powder diffraction pattern having characteristic peaks expressed in degrees 2-Theta at angles of about 4.8°±0.3°, about 8.9°±0.3°, about 11.3°±0.3°, about 14.9°±0.3°, about 18.0°±0.3°, about 19.4°±0.3°, about 21.2°±0.3° about 22.8°±0.3° and about 24.3°±0.3°.

In yet another embodiment, polymorph Ia is characterized by an X-ray powder diffraction pattern having 6-9 peaks expressed in degrees 2-Theta at angles selected from about 8.9°±0.2°, about 18.0°±0.2°, about 21.2°±0.2°, about 4.8°±0.2°, about 11.3°±0.2°, about 14.9°±0.2°, about 19.4°±0.2°, about 22.8°±0.2° and about 24.3°±0.2°. In a particular embodiment, polymorph Ia is characterized by an X-ray powder diffraction pattern having characteristic peaks expressed in degrees 2-Theta at angles of about 4.8°±0.2°, about 8.9°±0.2°, about 11.3°±0.2°, about 14.9°±0.2°, about 18.0°±0.2°, about 19.4°±0.2°, about 21.2°±0.2° about 22.8°±0.2° and about 24.3°±0.2°.

In still another embodiment, provided herein is a composition comprising crystalline tazobactam arginine characterized by an X-ray powder diffraction pattern having peaks expressed in degrees 2-Theta±0.3° at angles of 4.8°, 8.9°, 11.3°, 14.9°, 18.0°, 19.4°, 21.2°, and 22.8°.

In still another embodiment, provided herein is a composition comprising crystalline tazobactam arginine characterized by an X-ray powder diffraction pattern having peaks expressed in degrees 2-Theta±0.2° at angles of 4.8°, 8.9°, 11.3°, 14.9°, 18.0°, 19.4°, 21.2°, and 22.8°.

In still another embodiment, provided herein is a composition comprising crystalline tazobactam arginine characterized by an X-ray powder diffraction pattern having peaks expressed in degrees 2-Theta±0.1° at angles of 4.8°, 8.9°, 11.3°, 14.9°, 18.0°, 19.4°, 21.2°, and 22.8°.

In still another embodiment, provided herein is a composition comprising crystalline tazobactam arginine characterized by an X-ray powder diffraction pattern having peaks expressed in degrees 2-Theta at angles of about 4.8°, 8.9°, 11.3°, 14.9°, 18.0°, 19.4°, 21.2°, and 22.8°.

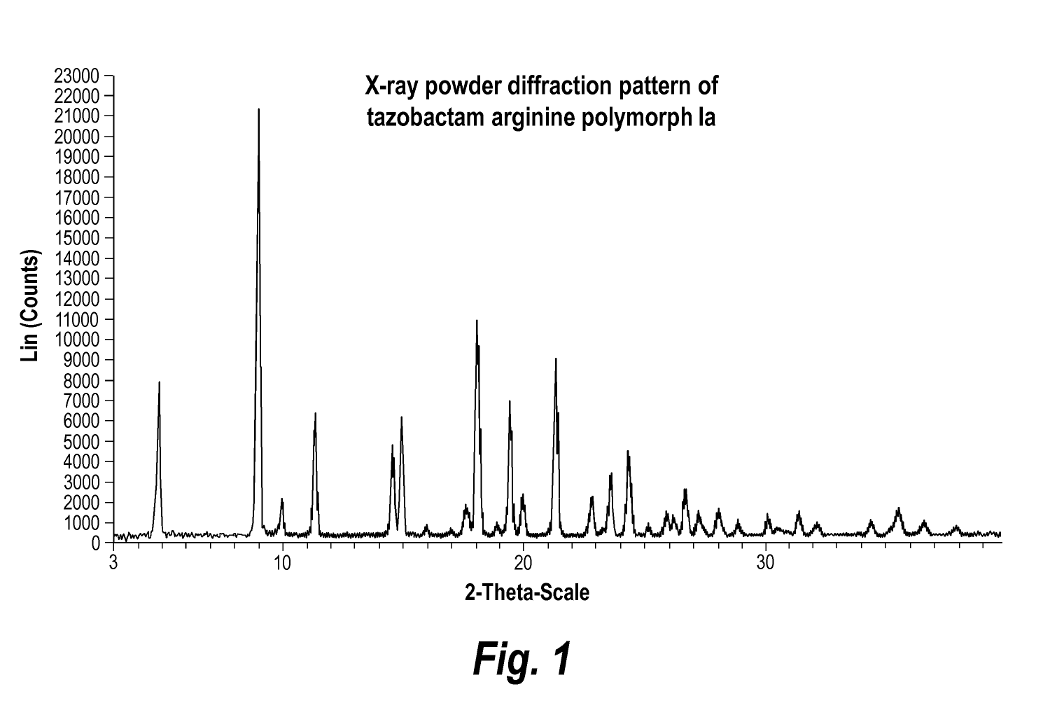

In one embodiment, polymorph Ia is characterized by an X-ray powder diffraction pattern having peaks substantially in accordance with FIG. 1. In another embodiment, polymorph Ia is characterized by an X-ray powder diffraction pattern having peaks substantially in accordance with Table 1.

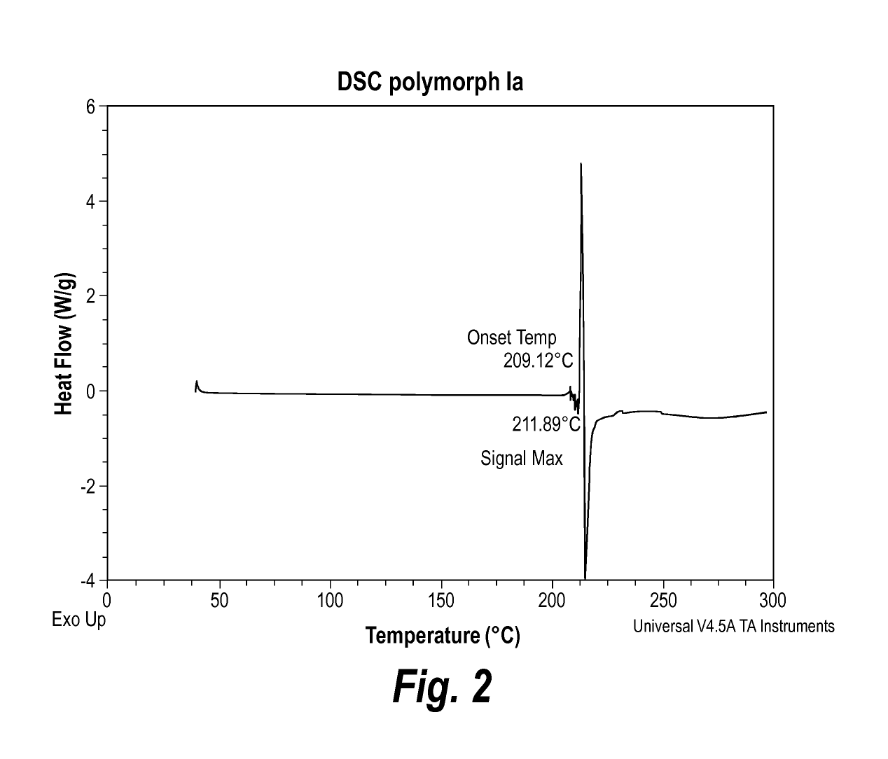

The compounds used in the combination therapies described herein may also be defined by their differential scanning calorimetry (DSC) thermograms. In one embodiment, polymorph Ia is characterized by a differential scanning calorimetry thermogram having a characteristic peak expressed in units of ° C. at a temperature of 209.2±3. In another embodiment, polymorph Ia is characterized by a differential scanning calorimetry thermogram having a characteristic peak expressed in units of ° C. in the range of about 209.2 to about 211.9. In a particular embodiment, polymorph Ia is characterized by a differential scanning calorimetry thermogram substantially in accordance with FIG. 2.

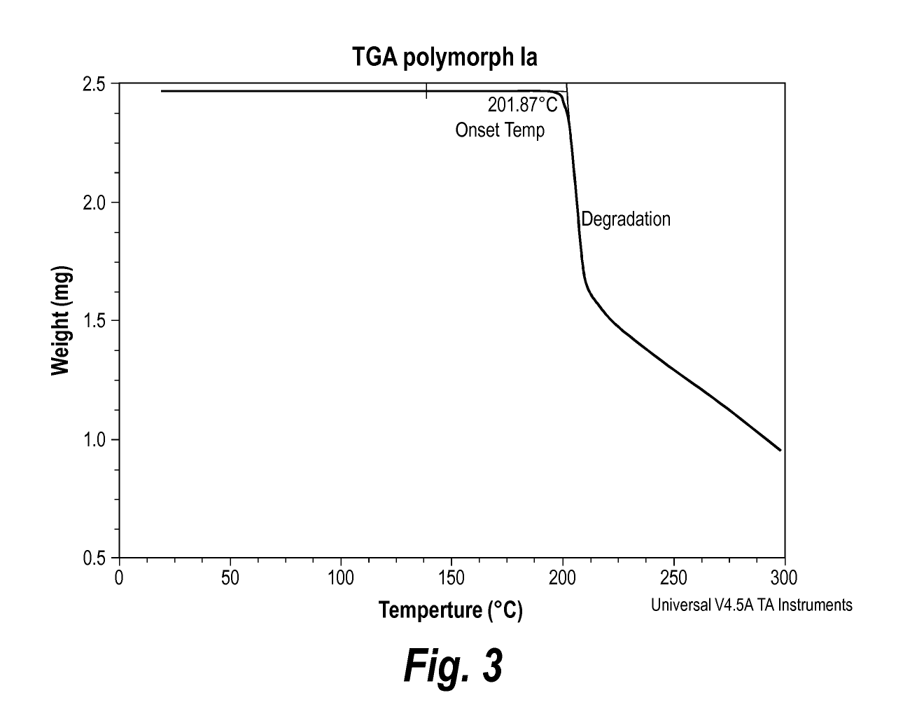

The compounds used in the combination therapies described herein can be also be defined by their thermogravimetry (TG) signals. In one embodiment, polymorph Ia is characterized by a thermogravimetry curve with an onset temperature of 201.8° C.±3° C. In another embodiment, polymorph Ia is characterized by a thermogravimetry curve with an onset temperature of about 201.8° C. In a particular embodiment, polymorph Ia is characterized by a thermogravimetry curve substantially in accordance with FIG. 3.

In certain embodiments, polymorph Ia may contain impurities. Non-limiting examples of impurities include undesired polymorph forms, or residual organic and inorganic molecules such as solvents, water or salts.

In another embodiment, polymorph Ia is substantially free from impurities. In another embodiment, polymorph Ia contains less than 10% by weight total impurities. In another embodiment, polymorph Ia contains less than 5% by weight total impurities. In another embodiment, polymorph Ia contains less than 1% by weight total impurities. In yet another embodiment, polymorph Ia contains less than 0.1% by weight total impurities.

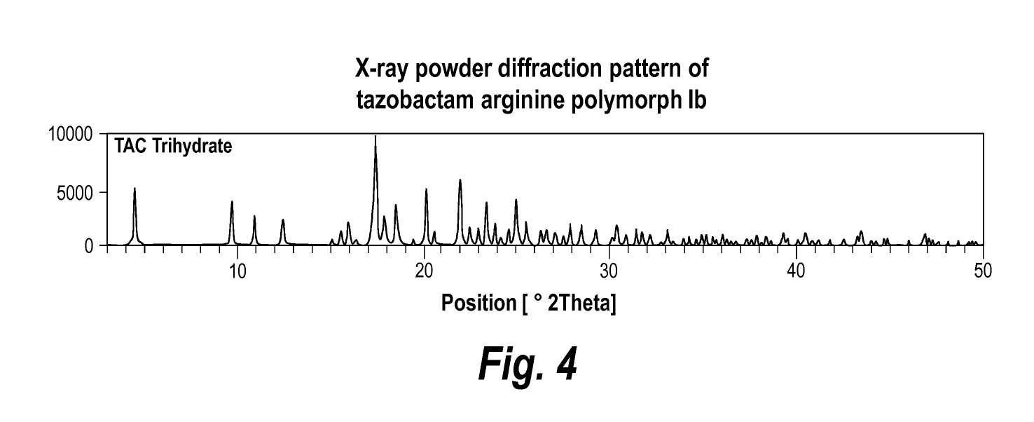

In another aspect, provided herein is crystalline tazobactam arginine polymorph Ib. In one embodiment, polymorph Ib is tazobactam arginine trihydrate. In another embodiment, crystalline tazobactam polymorph Ib is characterized by an X-ray powder diffraction pattern having peaks expressed in degrees 2-Theta at angles of about 4.4°±0.3°, about 9.7°±0.3°, about 17.3°±0.3°, about 20.2°±0.3°, and about 22.0°±0.3°. In a particular embodiment, polymorph Ib is characterized by an X-ray powder diffraction pattern having peaks substantially in accordance with FIG. 4.

In another aspect, provided herein is a combination comprising a beta-lactam compound and a composition comprising one or more compounds selected from amorphous tazobactam arginine, polymorph Ia and polymorph Ib. In one embodiment, the composition comprises one or more compounds selected from tazobactam arginine and polymorph Ia.

In certain embodiments, polymorph Ia is a crystalline solid substantially free of amorphous tazobactam arginine. As used herein, the term “substantially free of amorphous tazobactam arginine” means that the compound contains no significant amount of amorphous tazobactam arginine. In certain embodiments, at least about 95% by weight of crystalline polymorph Ia is present. In still other embodiments of the invention, at least about 99% by weight of crystalline polymorph Ia is present.

In another embodiment, polymorph Ia is substantially free from polymorph Ib. As used herein, the term “substantially free of polymorph Ib” means that the compound contains no significant amount of polymorph Ib. In certain embodiments, at least about 95% by weight of crystalline polymorph Ia is present. In still other embodiments of the invention, at least about 99% by weight of crystalline polymorph Ia is present.

### Beta-Lactam Compounds

A “beta-lactam compound” is a compound possessing one or more beta-lactam moieties, i.e.,

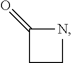

substituted one or more times as valency permits. In one embodiment, the beta-lactam compounds described herein are antibacterial compounds. In certain non-limiting embodiments the beta-lactam compounds described herein can be selected from the group consisting of penicillins, cephalosporins, carbapenems, and combinations thereof. In certain embodiments, the beta-lactam compounds are selected from the compounds listed in Table 2, and pharmaceutically acceptable isomers, salts, esters, hydrates, solvates, or combinations thereof. The following compounds are listed in Table 2:
(2S,5R,6R)-6-[(R)-2-(4-ethyl-2,3-dioxo-1-piperazinecarboxamido)-2-phenylacetamido]-3,3-dimethyl-7-oxo-4-thia-1-azabicyclo[3.2.0]heptane-2-carboxylic acid;
(2S,5R,6R)-3,3-dimethyl-7-oxo-6-(2-phenylacetamido)-4-thia-1-azabicyclo[3.2.0]heptane-2-carboxylic acid;
(5R,6S)-6-[(1R)-1-hydroxyethyl]-3-({2-[(iminomethyl)amino]ethyl}thio)-7-oxo-1-azabicyclo[3.2.0]hept-2-ene-2-carboxylic acid;
(5R,6S)-6-((R)-1-hydroxyethyl)-7-oxo-3-((R)-tetrahydrofuran-2-yl)-4-thia-1-azabicyclo[3.2.0]hept-2-ene-2-carboxylic acid;
(2S,5R,6R)-6-{[3-(2-chlorophenyl)-5-methyl-oxazole-4-carbonyl]amino}-3,3-dimethyl-7-oxo-4-thia-1-azabicyclo[3.2.0]heptane-2-carboxylic acid;
(6R,7R,Z)-7-(2-(2-aminothiazol-4-yl)-2-(2-carboxypropan-2-yloxyimino)acetamido)-8-oxo-3-(pyridinium-1-ylmethyl)-5-thia-1-aza-bicyclo[4.2.0]oct-2-ene-2-carboxylate;
(6R,7R,Z)-3-(acetoxymethyl)-7-(2-(2-aminothiazol-4-yl)-2-(methoxyimino)acetamido)-8-oxo-5-thia-1-azabicyclo[4.2.0]oct-2-ene-2-carboxylic acid;
(6R,7R)-7-[(2Z)-2-ethoxyimino-2-[5-(phosphono amino)-1,2,4-thiadiazol-3-yl]acetyl]amino]-3-[4-(1-methylpyridin-1-ium-4-yl)-1,3-thiazol-2-yl]sulfanyl]-8-oxo-5-thia-1-azabicyclo[4.2.0]oct-2-ene-2-carboxylate;
(6R,7R,Z)-7-(2-(2-aminothiazol-4-yl)-2-(methoxyimino)acetamido)-3-((1-methylpyrrolidinium-1-yl)methyl)-8-oxo-5-thia-1-aza-bicyclo[4.2.0]oct-2-ene-2-carboxylate;
(6R,7R)-3-{[(aminocarbonyl)oxy]methyl}-7-{[(2Z)-2-(2-furyl)-2-(methoxyimino)acetyl]amino}-8-oxo-5-thia-1-azabicyclo[4.2.0]oct-2-ene-2-carboxylic acid;
(6R,7R)-7-{[(2Z)-2-(2-amino-1,3-thiazol-4-yl)-2-(methoxyimino)acetyl]amino}-3-{[(2-methyl-5,6-dioxo-1,2,5,6-tetrahydro-1,2,4-triazin-3-yl)thio]methyl}-8-oxo-5-thia-1-azabicyclo[4.2.0]oct-2-ene-2-carboxylic acid;
(2S,5R,6R)-6-{[(2R)-2-amino-2-(4-hydroxyphenyl)-acetyl]amino}-3,3-dimethyl-7-oxo-4-thia-1-azabicyclo[3.2.0]heptane-2-carboxylic acid;
3-[5-(dimethylcarbamoyl)pyrrolidin-2-yl]sulfanyl-6-(1-hydroxyethyl)-4-methyl-7-oxo-1-azabicyclo[3.2.0]hept-2-ene-2-carboxylic acid;
(6R,7R)-3-[(5-amino-4-{[(2-aminoethyl) carbamoyl]amino}-1-methyl-1H-pyrazol-2-ium-2-yl)methyl]-7-({(2Z)-2-(5-amino-1,2,4-thiadiazol-3-yl)-2-[(1-carboxy-1-methylethoxy)imino]acetyl}amino)-8-oxo-5-thia-1-azabicyclo[4.2.0]oct-2-ene-2-carboxylate; and
5-amino-4-{[(2-aminoethyl)carbamoyl]amino}-2-{[(6R,7R)-7-({(2Z)-2-(5-amino-1,2,4-thiadiazol-3-yl)-2-[(1-carboxy-1-methylethoxy)imino]acetyl}amino)-2-carboxy-8-oxo-5-thia-1-azabicyclo[4.2.0]oct-2-en-3-yl]methyl}-1-methyl-1H-pyrazolium mono sulfate.

The skilled practitioner will recognize that the beta-lactam compounds described herein have one or more acidic moieties (e.g., carboxylic acid moieties) and/or one or more basic moieties (e.g., amine moieties). Said moieties may be protonated or deprotonated as a function of pKa or pKb of the moiety and the pH of the compound's environment. All salt forms resulting from the protonation or deprotonation of a beta-lactam compound are contemplated by the instant disclosure.

Any beta-lactam compound, exemplified by those listed above, can be used in the pharmaceutical compositions described herein.

The compound 5-amino-4-{[(2-aminoethyl)carbamoyl]amino}-2-{[(6R,7R)-7-({(2Z)-2-(5-amino-1,2,4-thiadiazol-3-yl)-2-[(1-carboxy-1-methylethoxy)imino]acetyl}amino)-2-carboxy-8-oxo-5-thia-1-azabicyclo[4.2.0]oct-2-en-3-yl]methyl}-1-methyl-1H-pyrazolium monosulfate (also known also as ceftolozane sulfate) is a cephalosporin compound (shown below), the synthesis of which is described in U.S. Pat. No. 7,129,232. As provided herein, ceftolozane can be in its free base form, or in the form of a pharmaceutically acceptable salt thereof, e.g., ceftolozane sulfate:

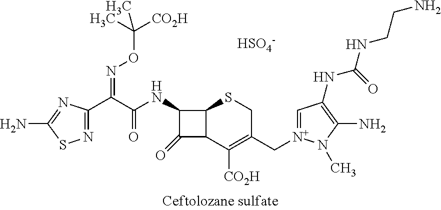

### Pharmaceutical Compositions

The term “pharmaceutical composition” includes preparations suitable for administration to mammals, e.g., humans. When the compounds of the present invention are administered as pharmaceuticals to mammals, e.g., humans, they can be given per se or as a pharmaceutical composition containing, for example, 0.1% to 99.9% (more preferably, 0.5 to 90%) of active ingredient in combination with a pharmaceutically acceptable carrier.

The pharmaceutical compositions described herein can be formulated to have any concentration desired (i.e., any concentration of crystalline tazobactam arginine, or a hydrate or solvate thereof, and any concentration of a beta-lactam compound). In some embodiments, the composition is formulated such that it comprises at least a therapeutically effective amount of both compounds (i.e., a therapeutically effective amount of the combination of crystalline tazobactam arginine, or a hydrate or solvate thereof, and the beta-lactam compound). In some embodiments, the composition is formulated such that it would not cause one or more unwanted side effects.

Pharmaceutical compositions include those suitable for oral, sublingual, nasal rectal, vaginal, topical, buccal and parenteral (including subcutaneous, intramuscular, and intravenous) administration, although the most suitable route will depend on the nature and severity of the condition being treated. The compositions may be conveniently presented in unit dosage form, and prepared by any of the methods well known in the art of pharmacy. In certain embodiments, the pharmaceutical composition is formulated for oral administration in the form of a pill, capsule, lozenge or tablet. In other embodiments, the pharmaceutical composition is in the form of a suspension.

Pharmaceutical compositions may additionally comprise excipients, stabilizers, pH adjusting additives (e.g., buffers) and the like. Non-limiting examples of these additives include sodium chloride, citric acid and L-arginine. For example, in the formulations of Example 2 and Example 3, the use of sodium chloride results in greater stability; L-arginine is used to adjust pH and to increase the solubility of ceftolozane; and citric acid is used prevent discoloration of the product, due to its ability to chelate metal ions.

The pharmaceutical compositions disclosed herein can be prepared via lyophilization (including, for example, co-lyophilization of more than one drug substances).

In a particular embodiment, the pharmaceutical compositions described herein are formulated for parenteral administration. In another particular embodiment, the pharmaceutical compositions described herein are formulated for administration by intravenous injection or infusion.

In one aspect, provided herein is a pharmaceutical composition comprising crystalline tazobactam arginine and a beta-lactam compound. In one embodiment, the beta-lactam compound is (6R,7R)-3-[(5-amino-4-{[(2-aminoethyl)carbamoyl]amino}-1-methyl-1H-pyrazol-2-ium-2-yl)methyl]-7-({(2Z)-2-(5-amino-1,2,4-thiadiazol-3-yl)-2-[(1-carboxy-1-methylethoxy)imino]acetyl}amino)-8-oxo-5-thia-1-azabicyclo[4.2.0]oct-2-ene-2-carboxylate, or a pharmaceutically acceptable isomer, salt, ester, hydrate, solvate, or combination thereof.

In another embodiment, the crystalline tazobactam arginine used in the combination therapies described herein is characterized by an X-ray powder diffraction pattern having one or more characteristic peaks expressed in degrees 2-Theta at angles of about 8.9°±0.3°, about 18.0°±0.3° and about 21.2°±0.3°. In yet another embodiment, the crystalline tazobactam arginine is characterized by an X-ray powder diffraction pattern having peaks expressed in degrees 2-Theta at angles of about 4.8°±0.3°, about 8.9°±0.3°, about 11.3°±0.3°, about 14.9°±0.3°, about 18.0°±0.3°, about 19.4°±0.3°, about 21.2°±0.3° about 22.8°±0.3° and about 24.3°±0.3°.

In another embodiment, the crystalline tazobactam arginine used in the combination therapies described herein is characterized by an X-ray powder diffraction pattern having one or more characteristic peaks expressed in degrees 2-Theta at angles of about 8.9°±0.2°, about 18.0°±0.2° and about 21.2°±0.2°. In yet another embodiment, the crystalline tazobactam arginine is characterized by an X-ray powder diffraction pattern having peaks expressed in degrees 2-Theta at angles of about 4.8°±0.2°, about 8.9°±0.2°, about 11.3°±0.2°, about 14.9°±0.2°, about 18.0°±0.2°, about 19.4°±0.2°, about 21.2°±0.2° about 22.8°±0.2° and about 24.3°±0.2°.

In still another embodiment, the crystalline tazobactam arginine is characterized by a differential scanning calorimetry thermogram having a characteristic peak expressed in units of ° C. at a temperature in the range of about 209.2 to about 211.9. In still another embodiment, the crystalline tazobactam arginine is characterized by a thermogravimetry curve with an onset temperature of about 201.9° C.

In a particular embodiment, the pharmaceutical composition comprises polymorph Ia and (6R,7R)-3-[(5-amino-4-{[(2-aminoethyl)carbamoyl]amino}-1-methyl-1H-pyrazol-2-ium-2-yl)methyl]-7-{(2Z)-2-(5-amino-1,2,4-thiadiazol-3-yl)-2-[(1-carboxy-1-methylethoxy)imino]acetyl}amino)-8-oxo-5-thia-1-azabicyclo[4.2.0]oct-2-ene-2-carboxylate, or a pharmaceutically acceptable isomer, salt, ester, hydrate, solvate, or combination thereof, and a pharmaceutically acceptable carrier or diluent. In a preferred embodiment, the pharmaceutical composition comprises polymorph Ia and 5-amino-4-{[(2-aminoethyl)carbamoyl]amino}-2-{[(6R,7R)-7-({(2Z)-2-(5-amino-1,2,4-thiadiazol-3-yl)-2-[(1-carboxy-1-methylethoxy)imino]acetyl}amino)-2-carboxy-8-oxo-5-thia-1-azabicyclo[4.2.0]oct-2-en-3-yl]methyl}-1-methyl-1H-pyrazolium monosulfate.

In another aspect, provided herein are pharmaceutical compositions prepared according to the following methods.

### Methods of Making Pharmaceutical Compositions

Provided herein is a method of making a pharmaceutical composition, comprising combining crystalline tazobactam arginine and a beta-lactam compound. In one embodiment, the method comprises the steps of: (1) preparing a mixture comprising crystalline tazobactam arginine and a beta-lactam compound; (2) preparing an aqueous solution from the mixture; and (3) lyophilizing the solution to obtain said pharmaceutical composition. In one embodiment, the method further comprises reconstituting the lyophilized mixture in an aqueous solvent, such that the resulting solution is suitable for parenteral administration.

The crystalline tazobactam arginine is characterized as described above. For example, in one embodiment of the method, the crystalline tazobactam arginine is characterized by an X-ray powder diffraction pattern having one or more characteristic peaks expressed in degrees 2-Theta at angles selected from about 8.9°±0.3°, about 18.0°±0.3° and about 21.2°±0.3°. In another embodiment, the crystalline tazobactam arginine is characterized by an X-ray powder diffraction pattern having one or more characteristic peaks expressed in degrees 2-Theta at angles of about 4.8°±0.3°, about 8.9°±0.3°, about 11.3°±0.3°, about 14.9°±0.3°, about 18.0°±0.3°, about 19.4°±0.3°, about 21.2°±0.3° about 22.8°±0.3° and about 24.3°±0.3°. In yet another embodiment, the crystalline tazobactam arginine is characterized by a differential scanning calorimetry thermogram having a characteristic peak expressed in units of ° C. at a temperature in the range of about 209.2 to about 211.9. In still another embodiment, the crystalline tazobactam arginine is characterized by a thermogravimetry curve with an onset temperature of about 201.9° C.

In another embodiment of the above method, and above embodiments, the beta-lactam compound is (6R,7R)-3-[(5-amino-4-{[(2-aminoethyl)carbamoyl]amino}-1-methyl-1H-pyrazol-2-ium-2-yl)methyl]-7-({(2Z)-2-(5-amino-1,2,4-thiadiazol-3-yl)-2-[(1-carboxy-1-methylethoxy)imino]acetyl}amino)-8-oxo-5-thia-1-azabicyclo[4.2.0]oct-2-ene-2-carboxylate, or a pharmaceutically acceptable isomer, salt, ester, hydrate, solvate, or combination thereof. In a particular embodiment, the beta-lactam compound is 5-amino-4-{[(2-aminoethyl)carbamoyl]amino}-2-{[(6R,7R)-7-({(2Z)-2-(5-amino-1,2,4-thiadiazol-3-yl)-2-[(1-carboxy-1-methylethoxy)imino]acetyl}amino)-2-carboxy-8-oxo-5-thia-1-azabicyclo[4.2.0]oct-2-en-3-yl]methyl}-1-methyl-1H-pyrazolium monosulfate.

In one embodiment of the method, and above embodiments, the molar ratio of crystalline tazobactam arginine to beta-lactam compound in the mixture is in the range of 1:3 to 3:1. In another embodiment, the molar ratio of crystalline tazobactam arginine to beta-lactam compound in the mixture is in the range of 1:2 to 2:1. In another embodiment, the molar ratio of crystalline tazobactam arginine to beta-lactam compound in the mixture is in the range of 1:0.9 to 0.9:1. In a particular embodiment, the ratio of crystalline tazobactam arginine to beta-lactam compound in the mixture is about 0.9:1. In another particular embodiment, the ratio of crystalline tazobactam arginine to beta-lactam compound in the mixture is about 1:2.

In some embodiments, the mixture of crystalline tazobactam arginine and ceftolozane further comprises one or more additives selected from the group consisting of L-arginine, citric acid, and sodium chloride. In one embodiment, the molar ratio of L-arginine to beta-lactam compound in the mixture is in the range of 4:1 to 1:4. In another embodiment, the molar ratio of L-arginine to beta-lactam compound in the mixture is in the range of 3:1 to 1:3. In another embodiment, the molar ratio of L-arginine to beta-lactam compound in the mixture is in the range of 2:1 to 1:2. In another embodiment, the molar ratio of L-arginine to beta-lactam compound in the mixture is in the range of about 4:1 to about 2:1. In a particular embodiment, the molar ratio of L-arginine to beta-lactam compound in the mixture is about 1.9:1.

In another embodiment of the method, the concentration of the beta-lactam compound in the aqueous solution is in the range of 0.01M-10M. In another embodiment, the concentration of the beta-lactam compound in the aqueous solution is in the range of 0.01M-1M. In a particular embodiment, the concentration of the beta-lactam compound in the aqueous solution is about 0.05M.

In still another embodiment of the method, the aqueous solution has a pH in the range of 5-7. In another embodiment, the aqueous solution has a pH in the range of 5.5-6.5. In a particular embodiment, the aqueous solution has a pH of about 6.3.

In another embodiment, ceftolozane (in free base or salt form, preferably hydrogen sulfate form) and tazobactam arginine are in a 2:1 (ceftolozane:tazobactam arginine) weight ratio, wherein the weight ratio is calculated based on the weight of ceftolozane in its free base, not salt, form. For example, a dose of the antibiotic composition comprising 300 mg ceftolozane hydrogen sulfate and 150 mg tazobactam arginine comprises an amount of ceftolozane hydrogen sulfate that corresponds to 300 mg of ceftolozane in its free base form.

In yet another embodiment, ceftolozane (in free base or salt form, preferably hydrogen sulfate form) and tazobactam arginine are in a 2:1 (ceftolozane:tazobactam) weight ratio, wherein the weight ratio is calculated based on the weights of ceftolozane and tazobactam in their free base, not salt, form. Accordingly, in a particular embodiment, the pharmaceutical composition comprises crystalline tazobactam arginine and ceftolozane sulfate in a ratio corresponding to one weight equivalent of tazobactam free base and two weight equivalents of ceftolozane free base.

### Methods of Treatment

Tazobactam arginine inhibits or decreases the activity of beta-lactamases (e.g., bacterial beta-lactamases), and can be combined with beta-lactam compounds (e.g., antibiotics), thereby broadening the spectrum of the beta-lactam compound and increasing the beta-lactam compound's efficacy against organisms that produce beta-lactamase. A compound or a composition possesses efficacy against an organism if it kills or weakens the organism, or inhibits or prevents reproduction the organism.

In one aspect, provided herein is a method for the treatment of bacterial infections in a mammal, comprising administering to said mammal a therapeutically effective amount of a pharmaceutical composition prepared according to the methods described herein. In another aspect, provided herein is a method for the treatment of bacterial infections in a mammal, comprising administering to said mammal a therapeutically effective amount of a crystalline tazobactam arginine and one or more beta-lactam compounds. In certain embodiments of the above methods, the bacterial infection is caused by an extended-spectrum beta-lactamase-producing organism. In certain embodiments, the bacterial infection is caused by an antibiotic-resistant organism.

In another aspect, provided herein is a method for the treatment of bacterial infections in a mammal, comprising administering to said mammal a therapeutically effective amount of a pharmaceutical composition comprising crystalline tazobactam arginine and one or more beta-lactam compounds. In one embodiment, the mammal is human. In another embodiment, the crystalline tazobactam arginine is polymorph Ia. In yet another embodiment, said one or more beta-lactam compounds are selected from the group consisting of penicillins, cephalosporins, carbapenems, and combinations thereof. In certain embodiments, the beta-lactam compound is selected from the compounds listed in Table 2, and pharmaceutically acceptable isomers, salts, esters, hydrates, solvates, or combinations thereof. In a particular embodiment, the beta-lactam compound is (6R,7R)-3-[(5-amino-4-{[(2-aminoethyl)carbamoyl]amino}-1-methyl-1H-pyrazol-2-ium-2-yl)methyl]-7-({(2Z)-2-(5-amino-1,2,4-thiadiazol-3-yl)-2-[(1-carboxy-1-methylethoxy)imino]acetyl}amino)-8-oxo-5-thia-1-azabicyclo[4.2.0]oct-2-ene-2-carboxylate, or a pharmaceutically acceptable isomer, salt, ester, hydrate, solvate, or combination thereof.

In another particular embodiment of the method, the pharmaceutical composition comprises polymorph Ia and 5-amino-4-{[(2-aminoethyl)carbamoyl]amino}-2-{[(6R,7R)-7-({(2Z)-2-(5-amino-1,2,4-thiadiazol-3-yl)-2-[(1-carboxy-1-methylethoxy)imino]acetyl}amino)-2-carboxy-8-oxo-5-thia-1-azabicyclo[4.2.0]oct-2-en-3-yl]methyl}-1-methyl-1H-pyrazolium monosulfate.

In another aspect, provided herein is a method for the treatment of bacterial infections in a mammal, comprising administering to said mammal a therapeutically effective amount of a pharmaceutical composition comprising an antibiotic and a crystalline tazobactam arginine compound (e.g., of the polymorph Ia solid form). The crystalline tazobactam arginine can be characterized by an X-ray powder diffraction pattern having peaks expressed in degrees 2-Theta at angles of about 4.8°±0.3°, about 8.9°±0.3°, about 11.3°±0.3°, about 14.9°±0.3°, about 18.0°±0.3°, about 19.4°±0.3°, about 21.2°±0.3° about 22.8°±0.3° and about 24.3°±0.3°. The crystalline tazobactam arginine can also be characterized by an X-ray powder diffraction pattern having peaks expressed in degrees 2-Theta at angles of about 4.8°±0.2°, about 8.9°±0.2°, about 11.3°±0.2°, about 14.9°±0.2°, about 18.0°±0.2°, about 19.4°±0.2°, about 21.2°±0.2° about 22.8°±0.2° and about 24.3°±0.2°.

Non-limiting examples of bacterial infections that can be treated by the methods of the invention include infections caused by: aerobic and facultative gram-positive microorganisms (e.g., Staphylococcus aureus, Enterococcus faecalis, Staphylococcus epidermidis, Streptococcus agalactiae, Streptococcus pneumonia, Streptococcus pyogenes, Viridans group streptococci), aerobic and facultative gram-negative microorganisms (e.g., Acinetobacter baumanii, Escherichia coli, Haemophilus influenza, Klebsiella pneumonia, Pseudomonas aeruginosa, Citrobacter koseri, Moraxella catarrhalis, Morganella morganii, Neisseria gonorrhoeae, Proteus mirabilis, Proteus vulgaris, Serratia marcescens, Providencia stuartii, Providencia rettgeri, Salmonella enterica), gram-positive anaerobes (Clostridium perfringens), and gram-negative anaerobes (e.g., Bacteroides fragilis group (e.g., B. fragilis, B. ovatus, B. thetaiotaomicron, and B. vulgates), Bacteroides distasonis, Prevotella melaninogenica).

In certain embodiments of the methods described herein, bacterial infection resulting from beta-lactamase-producing organisms are treated or controlled. Non-limiting examples of beta-lactamase-producing organisms include:

(1) ESBL (extended-spectrum beta-lactamase)-producing organisms selected from the group consisting of Enterobacteriaceae spp.: Escherichia coli, Klebsiella spp. (including K. pneumoniae and K. oxytoca), Proteus mirabilis, Proteus vulgaris, Enterobacter spp., Serratia spp., Citrobacter spp., Pseudomonas spp., Acinetobacter spp.) and Bacteroides spp.;

(2) CSBL (conventional-spectrum beta-lactamase)-producing organisms, known to those of skill in the art; and

(3) Inducible-AmpC-type beta-lactamases, such as Citrobacter spp., Serratia spp., Morganella morganii, Proteus vulgaris, and Enterobacter cloacae.

In certain embodiments of the methods described herein, bacterial infection is associated with one or more of the following conditions:

Appendicitis (complicated by rupture or abscess) and peritonitis caused by piperacillin-resistant beta-lactamase producing strains of Escherichia coli or the following members of the Bacteroides fragilis group: B. fragilis, B. ovatus, B. thetaiotaomicron, or B. vulgates;

Uncomplicated and complicated skin and skin structure infections, including cellulitis, cutaneous abscesses, and ischemic/diabetic foot infections caused by piperacillin-resistant, beta-lactamase producing strains of Staphylococcus aureus;

Postpartum endometritis or pelvic inflammatory disease caused by piperacillin-resistant, beta-lactamase producing strains of Escherichia coli;

Community-acquired pneumonia (moderate severity only) caused by piperacillin-resistant, beta-lactamase producing strains of Haemophilus influenza;

Nosocomial pneumonia (moderate to severe) caused by piperacillin-resistant, beta-lactamase producing strains of Staphylococcus aureus and by Acinetobacter baumanii, Haemophilus influenzae, Klebsiella pneumoniae, and Pseudomonas aeruginosa.

Complicated intra-abdominal infections; Complicated urinary tract infections (cUTIs); Acute Pyelonephritis; Systemic Inflammatory Response Syndrome (SIRS).

Also provided herein is the use of a crystalline tazobactam arginine, and hydrates and solvates thereof, in combination with one or more beta-lactam compounds, for the preparation of a medicament for the treatment of bacterial infection. The bacterial infection can result from either gram-negative or gram-positive organisms. In one embodiment, the crystalline tazobactam arginine is polymorph Ia. Polymorph Ia is characterized as described above. Said one or more beta-lactam compounds can be selected from the group consisting of penicillins, cephalosporins, carbapenems, and combinations thereof. In certain embodiments, said one or more beta-lactam compounds are selected from the compounds listed in Table 2, and pharmaceutically acceptable isomers, salts, esters, hydrates, solvates, or combinations thereof.

In one aspect, the invention provides crystalline tazobactam arginine and a beta-lactam compound for use in a method of treating a bacterial infection in a mammal. In one embodiment, the crystalline tazobactam arginine and beta-lactam compound are parenterally administered. Typically, the crystalline tazobactam arginine and beta-lactam compound are intravenously administered. In some embodiments, the crystalline tazobactam arginine and beta-lactam compound are administered as an infusion.

In one embodiment, the crystalline tazobactam arginine and beta-lactam compound are for use in a method of treating a bacterial infection in a mammal, wherein the bacterial infection is caused by an extended-spectrum beta-lactamase-producing organism. In another embodiment, the crystalline tazobactam arginine and beta-lactam compound are for use in a method of treating a bacterial infection in a mammal, wherein the bacterial infection is caused by an antibiotic-resistant organism. In a preferred embodiment, the crystalline tazobactam arginine and beta-lactam compound are for use in a method of treating a complicated urinary tract infection. In another preferred embodiment, the crystalline tazobactam arginine and beta-lactam compound are for use in a method of treating a complicated intra-abdominal infection. In a further preferred embodiment, the crystalline tazobactam arginine and beta-lactam compound are for use in a method of treating nosocomial pneumonia. The crystalline tazobactam arginine and beta-lactam compound may be for use in a method of treating ventilator acquired pneumonia or hospital acquired pneumonia.

In one preferred embodiment, the beta-lactam compound is (6R,7R)-3-[(5-amino-4-{[(2-aminoethyl)carbamoyl]amino}-1-methyl-1H-pyrazol-2-ium-2-yl)methyl]-7-({(2Z)-2-(5-amino-1,2,4-thiadiazol-3-yl)-2-[(1-carboxy-1-methylethoxy)imino]acetyl}amino)-8-oxo-5-thia-1-azabicyclo[4.2.0]oct-2-ene-2-carboxylate, or a pharmaceutically acceptable isomer, salt, ester, hydrate, solvate, or combination thereof. In a particularly preferred embodiment, the beta-lactam compound is 5-amino-4-{[(2-aminoethyl)carbamoyl]amino}-2-{[(6R,7R)-7-({(2Z)-2-(5-amino-1,2,4-thiadiazol-3-yl)-2-[(1-carboxy-1-methylethoxy)imino]acetyl}amino)-2-carboxy-8-oxo-5-thia-1-azabicyclo[4.2.0]oct-2-en-3-yl]methyl}-1-methyl-1H-pyrazolium monosulfate.

In one preferred embodiment, the crystalline tazobactam arginine is tazobactam arginine polymorph Ia. The crystalline tazobactam arginine may be characterized by an X-ray powder diffraction pattern having one or more characteristic peaks expressed in degrees 2-Theta at angles of about 8.9°±0.3°, about 18.0°±0.3° and about 21.2°±0.3°. The crystalline tazobactam arginine may be characterized by an X-ray powder diffraction pattern having peaks expressed in degrees 2-Theta at angles of about 4.8°±0.3°, about 8.9°±0.3°, about 11.3°±0.3°, about 14.9°±0.3°, about 18.0°±0.3°, about 19.4°±0.3°, about 21.2°±0.3° about 22.8°±0.3° and about 24.3°±0.3°. In some embodiments, the crystalline tazobactam arginine is characterized by a differential scanning calorimetry thermogram having a characteristic peak expressed in units of ° C. at a temperature in the range of about 209.2 to about 211.9. The crystalline tazobactam arginine may be characterized by a thermogravimetry curve with an onset temperature of about 201.9° C.

In the most preferred embodiments, the beta-lactam compound is 5-amino-4-{[(2-aminoethyl)carbamoyl]amino}-2-{[(6R,7R)-7-({(2Z)-2-(5-amino-1,2,4-thiadiazol-3-yl)-2-[(1-carboxy-1-methylethoxy)imino]acetyl}amino)-2-carboxy-8-oxo-5-thia-1-azabicyclo[4.2.0]oct-2-en-3-yl]methyl}-1-methyl-1H-pyrazolium monosulfate and the crystalline tazobactam arginine is tazobactam arginine polymorph Ia.

In one aspect, the invention provides crystalline tazobactam arginine for use in a method of treating a bacterial infection in a mammal, comprising administration of crystalline tazobactam arginine in combination with a beta-lactam compound. In one embodiment, the crystalline tazobactam arginine and/or beta-lactam compound is parenterally administered. Typically, the crystalline tazobactam arginine and/or beta-lactam compound is intravenously administered. In some embodiments, the crystalline tazobactam arginine and/or beta-lactam compound is administered as an infusion. In one embodiment, both the crystalline tazobactam arginine and beta-lactam compound are parenterally administered. In one embodiment, both the crystalline tazobactam arginine and beta-lactam compound are intravenously administered. In another embodiment, both the crystalline tazobactam arginine and beta-lactam compound are administered as an infusion.

In one embodiment, the crystalline tazobactam arginine is for use in a method of treating a bacterial infection in a mammal, wherein the bacterial infection is caused by an extended-spectrum beta-lactamase-producing organism. In another embodiment, the crystalline tazobactam arginine is for use in a method of treating a bacterial infection in a mammal, wherein the bacterial infection is caused by an antibiotic-resistant organism. In a preferred embodiment, the crystalline tazobactam arginine is for use in a method of treating a complicated urinary tract infection. In another preferred embodiment, the crystalline tazobactam arginine is for use in a method of treating a complicated intra-abdominal infection. In a further preferred embodiment, the crystalline tazobactam arginine is for use in a method of treating nosocomial pneumonia. The crystalline tazobactam arginine may be for use in a method of treating ventilator acquired pneumonia or hospital acquired pneumonia.

In one preferred embodiment, the beta-lactam compound is (6R,7R)-3-[(5-amino-4-{[(2-aminoethyl)carbamoyl]amino}-1-methyl-1H-pyrazol-2-ium-2-yl)methyl]-7-({(2Z)-2-(5-amino-1,2,4-thiadiazol-3-yl)-2-[(1-carboxy-1-methylethoxy)imino]acetyl}amino)-8-oxo-5-thia-1-azabicyclo[4.2.0]oct-2-ene-2-carboxylate, or a pharmaceutically acceptable isomer, salt, ester, hydrate, solvate, or combination thereof. In a particularly preferred embodiment, the beta-lactam compound is 5-amino-4-{[(2-aminoethyl)carbamoyl]amino}-2-{[(6R,7R)-7-({(2Z)-2-(5-amino-1,2,4-thiadiazol-3-yl)-2-[(1-carboxy-1-methylethoxy)imino]acetyl}amino)-2-carboxy-8-oxo-5-thia-1-azabicyclo[4.2.0]oct-2-en-3-yl]methyl}-1-methyl-1H-pyrazolium mono sulfate.

In one preferred embodiment, the crystalline tazobactam arginine is tazobactam arginine polymorph Ia. The crystalline tazobactam arginine may be characterized by an X-ray powder diffraction pattern having one or more characteristic peaks expressed in degrees 2-Theta at angles of about 8.9°±0.3°, about 18.0°±0.3° and about 21.2°±0.3°. The crystalline tazobactam arginine may be characterized by an X-ray powder diffraction pattern having peaks expressed in degrees 2-Theta at angles of about 4.8°±0.3°, about 8.9°±0.3°, about 11.3°±0.3°, about 14.9°±0.3°, about 18.0°±0.3°, about 19.4°±0.3°, about 21.2°±0.3° about 22.8°±0.3° and about 24.3°±0.3°. In some embodiment, the crystalline tazobactam arginine is characterized by a differential scanning calorimetry thermogram having a characteristic peak expressed in units of ° C. at a temperature in the range of about 209.2 to about 211.9. The crystalline tazobactam arginine may be characterized by a thermogravimetry curve with an onset temperature of about 201.9° C.

In the most preferred embodiments, the beta-lactam compound is 5-amino-4-{[(2-aminoethyl)carbamoyl]amino}-2-{[(6R,7R)-7-({(2Z)-2-(5-amino-1,2,4-thiadiazol-3-yl)-2-[(1-carboxy-1-methylethoxy)imino]acetyl}amino)-2-carboxy-8-oxo-5-thia-1-azabicyclo[4.2.0]oct-2-en-3-yl]methyl}-1-methyl-1H-pyrazolium monosulfate and the crystalline tazobactam arginine is tazobactam arginine polymorph Ia.

In one aspect, the invention provides a beta-lactam compound for use in a method of treating a bacterial infection in a mammal, comprising administration of a beta-lactam compound in combination with crystalline tazobactam arginine. In one embodiment, the beta-lactam compound and/or crystalline tazobactam arginine is parenterally administered. Typically, the beta-lactam compound and/or crystalline tazobactam arginine is intravenously administered. In some embodiments, the beta-lactam compound and/or crystalline tazobactam arginine is administered as an infusion. In one embodiment, both the beta-lactam compound and crystalline tazobactam arginine are parenterally administered. In one embodiment, both the beta-lactam compound and crystalline tazobactam arginine are intravenously administered. In another embodiment, both the beta-lactam compound and crystalline tazobactam arginine are intravenously administered as an infusion.

In one embodiment, the beta-lactam compound is for use in a method of treating a bacterial infection in a mammal, wherein the bacterial infection is caused by an extended-spectrum beta-lactamase-producing organism. In another embodiment, the beta-lactam compound is for use in a method of treating a bacterial infection in a mammal, wherein the bacterial infection is caused by an antibiotic-resistant organism. In a preferred embodiment, the beta-lactam compound is for use in a method of treating a complicated urinary tract infection. In another preferred embodiment, the beta-lactam compound is for use in a method of treating a complicated intra-abdominal infection. In a further preferred embodiment, the beta-lactam compound is for use in a method of treating nosocomial pneumonia. The beta-lactam compound may be for use in a method of treating ventilator acquired pneumonia or hospital acquired pneumonia.

In one preferred embodiment, the beta-lactam compound is (6R,7R)-3-[(5-amino-4-{[(2-aminoethyl)carbamoyl]amino}-1-methyl-1H-pyrazol-2-ium-2-yl)methyl]-7-({(2Z)-2-(5-amino-1,2,4-thiadiazol-3-yl)-2-[(1-carboxy-1-methylethoxy)imino]acetyl}amino)-8-oxo-5-thia-1-azabicyclo[4.2.0]oct-2-ene-2-carboxylate, or a pharmaceutically acceptable isomer, salt, ester, hydrate, solvate, or combination thereof. In a particularly preferred embodiment, the beta-lactam compound is 5-amino-4-{[(2-aminoethyl)carbamoyl]amino}-2-{[(6R,7R)-7-({(2Z)-2-(5-amino-1,2,4-thiadiazol-3-yl)-2-[(1-carboxy-1-methylethoxy)imino]acetyl}amino)-2-carboxy-8-oxo-5-thia-1-azabicyclo[4.2.0]oct-2-en-3-yl]methyl}-1-methyl-1H-pyrazolium monosulfate.

In one preferred embodiment, the crystalline tazobactam arginine is tazobactam arginine polymorph Ia. The crystalline tazobactam arginine may be characterized by an X-ray powder diffraction pattern having one or more characteristic peaks expressed in degrees 2-Theta at angles of about 8.9°±0.3°, about 18.0°±0.3° and about 21.2°±0.3°. The crystalline tazobactam arginine may be characterized by an X-ray powder diffraction pattern having peaks expressed in degrees 2-Theta at angles of about 4.8°±0.3°, about 8.9°±0.3°, about 11.3°±0.3°, about 14.9°±0.3°, about 18.0°±0.3°, about 19.4°±0.3°, about 21.2°±0.3° about 22.8°±0.3° and about 24.3°±0.3°. In some embodiment, the crystalline tazobactam arginine is characterized by a differential scanning calorimetry thermogram having a characteristic peak expressed in units of ° C. at a temperature in the range of about 209.2 to about 211.9. The crystalline tazobactam arginine may be characterized by a thermogravimetry curve with an onset temperature of about 201.9° C.

In the most preferred embodiments, the beta-lactam compound is 5-amino-4-{[(2-aminoethyl)carbamoyl]amino}-2-{[(6R,7R)-7-({(2Z)-2-(5-amino-1,2,4-thiadiazol-3-yl)-2-[(1-carboxy-1-methylethoxy)imino]acetyl}amino)-2-carboxy-8-oxo-5-thia-1-azabicyclo[4.2.0]oct-2-en-3-yl]methyl}-1-methyl-1H-pyrazolium monosulfate and the crystalline tazobactam arginine is tazobactam arginine polymorph Ia.

In one aspect, the invention provides crystalline tazobactam arginine and a beta-lactam compound as a combined preparation for simultaneous, separate or sequential use in a method of treating a bacterial infection in a mammal. In one embodiment, the crystalline tazobactam arginine and beta-lactam compound are parenterally administered. Typically, the crystalline tazobactam arginine and beta-lactam compound are intravenously administered. In some embodiments, the crystalline tazobactam arginine and beta-lactam compound are administered as an infusion.

In one embodiment, the crystalline tazobactam arginine and beta-lactam compound are for use in a method of treating a bacterial infection in a mammal, wherein the bacterial infection is caused by an extended-spectrum beta-lactamase-producing organism. In another embodiment, the crystalline tazobactam arginine and beta-lactam compound are for use in a method of treating a bacterial infection in a mammal, wherein the bacterial infection is caused by an antibiotic-resistant organism. In a preferred embodiment, the crystalline tazobactam arginine and beta-lactam compound are for use in a method of treating a complicated urinary tract infection. In another preferred embodiment, the crystalline tazobactam arginine and beta-lactam compound are for use in a method of treating a complicated intra-abdominal infection. In a further preferred embodiment, the crystalline tazobactam arginine and beta-lactam compound are for use in a method of treating nosocomial pneumonia. The crystalline tazobactam arginine and beta-lactam compound may be for use in a method of treating ventilator acquired pneumonia or hospital acquired pneumonia.

In one preferred embodiment, the beta-lactam compound is (6R,7R)-3-[(5-amino-4-{[(2-aminoethyl)carbamoyl]amino}-1-methyl-1H-pyrazol-2-ium-2-yl)methyl]-7-({(2Z)-2-(5-amino-1,2,4-thiadiazol-3-yl)-2-[(1-carboxy-1-methylethoxy)imino]acetyl}amino)-8-oxo-5-thia-1-azabicyclo[4.2.0]oct-2-ene-2-carboxylate, or a pharmaceutically acceptable isomer, salt, ester, hydrate, solvate, or combination thereof. In a particularly preferred embodiment, the beta-lactam compound is 5-amino-4-{[(2-aminoethyl)carbamoyl]amino}-2-{[(6R,7R)-7-({(2Z)-2-(5-amino-1,2,4-thiadiazol-3-yl)-2-[(1-carboxy-1-methylethoxy)imino]acetyl}amino)-2-carboxy-8-oxo-5-thia-1-azabicyclo[4.2.0]oct-2-en-3-yl]methyl}-1-methyl-1H-pyrazolium monosulfate.

In one preferred embodiment, the crystalline tazobactam arginine is tazobactam arginine polymorph Ia. The crystalline tazobactam arginine may be characterized by an X-ray powder diffraction pattern having one or more characteristic peaks expressed in degrees 2-Theta at angles of about 8.9°±0.3°, about 18.0°±0.3° and about 21.2°±0.3°. The crystalline tazobactam arginine may be characterized by an X-ray powder diffraction pattern having peaks expressed in degrees 2-Theta at angles of about 4.8°±0.3°, about 8.9°±0.3°, about 11.3°±0.3°, about 14.9°±0.3°, about 18.0°±0.3°, about 19.4°±0.3°, about 21.2°±0.3° about 22.8°±0.3° and about 24.3°±0.3°. In some embodiment, the crystalline tazobactam arginine is characterized by a differential scanning calorimetry thermogram having a characteristic peak expressed in units of ° C. at a temperature in the range of about 209.2 to about 211.9. The crystalline tazobactam arginine may be characterized by a thermogravimetry curve with an onset temperature of about 201.9° C.

In the most preferred embodiments, the beta-lactam compound is 5-amino-4-{[(2-aminoethyl)carbamoyl]amino}-2-{[(6R,7R)-7-({(2Z)-2-(5-amino-1,2,4-thiadiazol-3-yl)-2-[(1-carboxy-1-methylethoxy)imino]acetyl}amino)-2-carboxy-8-oxo-5-thia-1-azabicyclo[4.2.0]oct-2-en-3-yl]methyl}-1-methyl-1H-pyrazolium monosulfate and the crystalline tazobactam arginine is tazobactam arginine polymorph Ia.

In one aspect, the invention provides crystalline tazobactam arginine and a beta-lactam compound for use in therapy. In one embodiment, the crystalline tazobactam arginine and beta-lactam compound are parenterally administered. Typically, the crystalline tazobactam arginine and beta-lactam compound are intravenously administered. In some embodiments, the crystalline tazobactam arginine and beta-lactam compound are administered as an infusion.

In one preferred embodiment, the beta-lactam compound is (6R,7R)-3-[(5-amino-4-{[(2-aminoethyl)carbamoyl]amino}-1-methyl-1H-pyrazol-2-ium-2-yl)methyl]-7-({(2Z)-2-(5-amino-1,2,4-thiadiazol-3-yl)-2-[(1-carboxy-1-methylethoxy)imino]acetyl}amino)-8-oxo-5-thia-1-azabicyclo[4.2.0]oct-2-ene-2-carboxylate, or a pharmaceutically acceptable isomer, salt, ester, hydrate, solvate, or combination thereof. In a particularly preferred embodiment, the beta-lactam compound is 5-amino-4-{[(2-aminoethyl)carbamoyl]amino}-2-{[(6R,7R)-7-({(2Z)-2-(5-amino-1,2,4-thiadiazol-3-yl)-2-[(1-carboxy-1-methylethoxy)imino]acetyl}amino)-2-carboxy-8-oxo-5-thia-1-azabicyclo[4.2.0]oct-2-en-3-yl]methyl}-1-methyl-1H-pyrazolium monosulfate.

In one preferred embodiment, the crystalline tazobactam arginine is tazobactam arginine polymorph Ia. The crystalline tazobactam arginine may be characterized by an X-ray powder diffraction pattern having one or more characteristic peaks expressed in degrees 2-Theta at angles of about 8.9°±0.3°, about 18.0°±0.3° and about 21.2°±0.3°. The crystalline tazobactam arginine may be characterized by an X-ray powder diffraction pattern having peaks expressed in degrees 2-Theta at angles of about 4.8°±0.3°, about 8.9°±0.3°, about 11.3°±0.3°, about 14.9°±0.3°, about 18.0°±0.3°, about 19.4°±0.3°, about 21.2°±0.3° about 22.8°±0.3° and about 24.3°±0.3°. In some embodiments, the crystalline tazobactam arginine is characterized by a differential scanning calorimetry thermogram having a characteristic peak expressed in units of ° C. at a temperature in the range of about 209.2 to about 211.9. The crystalline tazobactam arginine may be characterized by a thermogravimetry curve with an onset temperature of about 201.9° C.

In the most preferred embodiments, the beta-lactam compound is 5-amino-4-{[(2-aminoethyl)carbamoyl]amino}-2-{[(6R,7R)-7-({(2Z)-2-(5-amino-1,2,4-thiadiazol-3-yl)-2-[(1-carboxy-1-methylethoxy)imino]acetyl}amino)-2-carboxy-8-oxo-5-thia-1-azabicyclo[4.2.0]oct-2-en-3-yl]methyl}-1-methyl-1H-pyrazolium monosulfate and the crystalline tazobactam arginine is tazobactam arginine polymorph Ia.

As used herein, “treating”, “treat” or “treatment” describes the management and care of a patient for the purpose of combating a disease, condition, or disorder and includes the administration of a pharmaceutical composition of the present invention to alleviate the symptoms or complications of a disease, condition or disorder, or to eliminate the disease, condition or disorder. The term “treat” can also include treatment of a cell in vitro or an animal model.

By a “therapeutically effective amount” of a compound of the invention is meant a sufficient amount of the compound to treat the disorder (e.g., bacterial infection). The specific therapeutically effective amount that is required for the treatment of any particular patient or organism (e.g., a mammal) will depend upon a variety of factors including the disorder being treated and the severity of the disorder; the activity of the specific compound or composition employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts (see, for example, Goodman and Gilman's, “The Pharmacological Basis of Therapeutics”, Tenth Edition, A. Gilman, J. Hardman and L. Limbird, eds., McGraw-Hill Press, 155-173, 2001, which is incorporated herein by reference in its entirety). The therapeutically effective amount for a given situation can be readily determined by routine experimentation and is within the skill and judgment of the ordinary clinician.

### Assays

Provided herein is a method for detecting or identifying an agent that will inhibit one or more beta-lactamase-producing organisms, said method comprising combining:

(a) a test agent;

(b) a composition comprising one or more beta-lactamase-producing organisms; and

(c) a beta-lactamase inhibitor; and detecting or measuring a change in the activity of the beta-lactamase-producing organisms, wherein a decrease in the activity of the beta-lactamase-producing organisms indicates that the test agent inhibits the beta-lactamase-producing organisms.

As used in the above method, “activity” refers to the ability of the beta-lactamase-producing organism to reproduce and/or infect another organism, or “activity” refers to the presence of an indicator of the ability of the beta-lactamase-producing organism to reproduce and/or infect another organism. Methods for detecting and/or measuring changes in the activity of beta-lactamase-producing organisms are known to those of skill in the art.

In another aspect, provided herein is a method of determining the susceptibility of a beta-lactamase-producing organism to a composition comprising a beta-lactam compound and a beta-lactamase inhibitor. The in vitro activity of compositions of the subject invention may be assessed by standard testing procedures. Non-limiting examples of such a procedure include the Kirby-Bauer method, the Stokes test, the E-test, broth dilution and agar dilution for determination of minimum inhibitory concentration (MIC), as described in “Approved Standard. Methods for Dilution Antimicrobial Susceptibility Tests for Bacteria that Grow Aerobically,” 3.sup.rd ed., published 1993 by the National Committee for Clinical Laboratory standards, Villanova, Pa., USA. In certain embodiments, the methods described herein are performed using automation (e.g., Siemens+MicroScan Systems).

In one embodiment of the above methods, the beta-lactamase inhibitor is tazobactam arginine. In a preferred embodiment, the beta-lactamase inhibitor is tazobactam arginine polymorph Ia.

The test agent can be selected from the group consisting of penicillins, cephalosporins, carbapenems, and combinations thereof. In some embodiments, the test agent is selected from the compounds listed in Table 2, and pharmaceutically acceptable isomers, salts, esters, hydrates, solvates, or combinations thereof.

In certain embodiments of the methods described herein, beta-lactamase-producing organisms are selected from the group comprising: (1) ESBL (extended-spectrum beta-lactamase)-producing organisms selected from the group consisting of Enterobacteriaceae spp.: Escherichia coli, Klebsiella spp. (including K. pneumoniae and K. oxytoca), Proteus mirabilis, Proteus vulgaris, Enterobacter spp., Serratia spp., Citrobacter spp.) and Bacteroides spp.;

(2) CSBL (conventional-spectrum beta-lactamase)-producing organisms, known to those of skill in the art; and

(3) Inducible-AmpC-type beta-lactamases, such as Citrobacter spp., Serratia spp., Morganella morganii, Proteus vulgaris, and Enterobacter cloacae.

### Instrumentation and Methods

I. X-Ray Powder Diffraction (XRPD) experiments were performed using a Bruker D8 Advance X-ray powder diffractometer utilizing a zero return silicon plate, a step size of 0.01°, a step time of 0.3 sec/step, Cu/Kα radiation, tube power of 40 kV/40 mA, a nickel filter, and a LynxEye high speed detector. A suitable amount of sample was placed directly on the sample holder, pressed flat to smooth, and analyzed from 3°-40° 20 using Bragg-Brentano optics. Analysis was started immediately following sample preparation.
II. Differential Scanning calorimetry (DSC) experiments were performed on a TA Instruments Q100 instrument. A temperature range of 40° C. to 300° C. with a ramp rate of 10° C./minute was utilized. Approximately 1.0 mg of sample was weighed into a tared aluminum sample pan and sealed hermetically. A small hole was pushed into the cover of the sample pan to allow for pressure release.
III. Thermo Gravemetric Analysis (TGA) experiments were performed on a TA Instruments 5000 instrument from 20 to 300° C. with a heating rate of 10° C./minute for all samples.

### EXAMPLES

### Example 1

### Preparation of Tazobactam Arginine Crystalline Polymorph Ia

Tazobactam arginine amorphous (1.00 g) was dissolved in 10.0 mL of deionized water. 30 mL of acetone was added to the aqueous solution by drop-wise addition. The mixture was allowed to sit overnight at ambient temperature, resulting in white fine needles. After filtration and vacuum drying for 4 hours, tazobactam arginine polymorph Ia (516 mg) was obtained. The XRPD spectrum of the tazobactam arginine polymorph Ia is depicted in FIG. 1.

### Example 2

### Preparation of Pharmaceutical Composition Using Tazobactam Arginine Polymorph Ia and Ceftolozane

A mixture is prepared comprising: tazobactam arginine polymorph Ia and ceftolozane in a molar ratio in the range of 1:2 to 2:1; L-arginine, such that the molar ratio of L-arginine to ceftolozane is in the range of 4:1 to 1:4; citric acid, such that the pH of an aqueous solution of the mixture is in the range of 5-7; and sodium chloride, such that the concentration of sodium chloride in an aqueous solution of the mixture is in the range of 0.1M 1 M. The mixture is dissolved in deionized water, such that the molar ratio of ceftolozane in the aqueous solution is in the range of 0.01M-10M. The resulting aqueous solution is then lyophilized to afford the title pharmaceutical composition.

### Example 3

### Stability of Formulations of Ceftolozane and Solid Forms of Tazobactam

Formulations A-D of Table 3 were prepared as follows:

Formulation A: 1.237 g (1.5 mmol) of 90% ceftolozane sulfate, 0.62 g (3.56 mmol) of L-arginine, 0.022 g (0.115 mmol) of citric acid, 0.49 g (8.39 mmol) of NaCl was dissolved in 30 mL of water (final pH 5.81), then filtered through a 0.2 μm membrane, and lyophilized 24 hr to obtain an off-white powder, 2.2 g. A 480 mg portion was used for stability testing at 25° C. (60% RH).
Formulation B: 1.237 g (1.5 mmol) of 90% ceftolozane sulfate, 0.93 g (5.34 mmol) of L-arginine, 0.022 g (0.115 mmol) of citric acid, 0.50 g (1.67 mmol) of tazobactam acid, and 0.49 g (8.39 mmol) of NaCl was dissolved in 30 mL of water (final pH 6.72), then filtered through a 0.2 μm membrane, and lyophilized 24 hr to obtain an off-white powder, 3.22 g. A 490 mg portion was used for stability testing at 25° C. (60% RH).
Formulation C: 1.237 g (1.5 mmol) of 90% ceftolozane sulfate, 0.62 g (3.56 mmol) of L-arginine, 0.022 g (0.115 mmol) of citric acid, and 0.49 g (8.39 mmol) of NaCl was dissolved in 30 mL of water (resulting pH 6.34), then added 0.79 g (1.67 mmol) of tazobactam arginine polymorph Ia and stirred to dissolve (final pH 6.30), filtered through a 0.2 μm membrane, and lyophilized 24 hr to obtain an off-white powder, 3.10 g. A 510 mg portion was used for stability testing at 25° C. (60% RH).
Formulation D: 1.0 g of Formulation A (0.7 mmol ceftolozane sulfate; 1.67 mmol L-arginine), and 0.21 g (0.65 mmol) tazobactam sodium was dissolved in 20 mL of water (final pH 5.89), then filtered through a 0.2 μm membrane, and lyophilized 24 hr to obtain an off white-powder, 1.074 g. A 195 mg portion was tested for stability at 25° C. (60% RH).

The above formulations were analyzed by HPLC at the following time points: T0: (Immediately after lyophilization); T1 (After one month at 25° C. and 60% relative humidity); and T2 (After three months at 25° C. and 60% relative humidity).

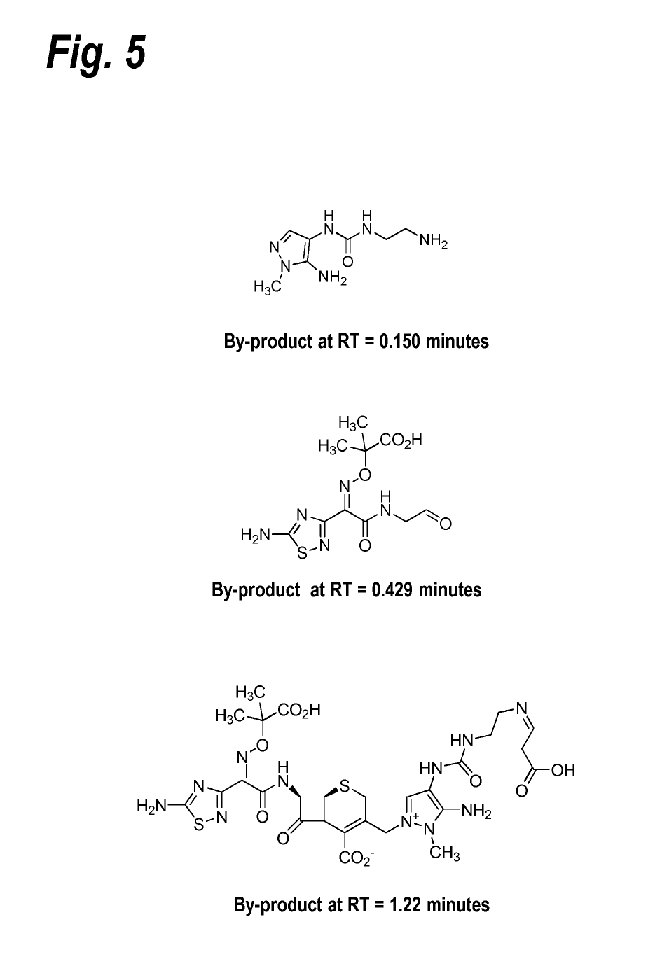

Of the three tazobactam-containing formulations (B, C and D), formulation D (containing tazobactam sodium) exhibited the highest degree of ceftolozane decomposition at T2. Formulation B (containing tazobactam acid and L-arginine) exhibited less ceftolozane decomposition than formulation D, and formulation C (containing tazobactam arginine polymorph Ia) exhibited significantly less ceftolozane decomposition than formulation B. Formulation C also exhibited significantly lower amounts of by-products having retention times of 0.150, 0.429 and 1.22 minutes, shown in FIG. 5. These results are summarized in Table 4.

TABLE 1
XRPD Scanning Data of Tazobactam
Arginine Polymorph Ia (FIG. 1)

| Chord | D (Obs. | Inten- | Max | Inten- | I. |
|---|---|---|---|---|---|
| Mid. | Max) | sity % | Int. | sity | Breadth |
| 2-Theta ° | Angstrom | % | Cps | Count | 2-Theta ° |
| 4.818 | 18.27951 | 33.5 | 130 | 7043 | 0.166 |
| 8.978 | 9.83463 | 100.0 | 364 | 21035 | 0.174 |
| 9.916 | 8.90757 | 8.7 | 32.3 | 1832 | 0.168 |
| 11.301 | 7.81865 | 27.8 | 104 | 5844 | 0.167 |
| 14.521 | 6.09321 | 20.2 | 75.5 | 4251 | 0.108 |
| 14.902 | 5.93864 | 27.8 | 102 | 5850 | 0.162 |
| 15.93 | 5.56039 | 1.9 | 7.2 | 394 | 0.148 |
| 16.947 | 5.23254 | 1.2 | 4.96 | 253 | 0.169 |
| 17.581 | 5.04332 | 6.8 | 24.8 | 1429 | 0.182 |
| 18.046 | 4.91261 | 48.7 | 184 | 10242 | 0.189 |
| 18.863 | 4.70152 | 2.6 | 9.41 | 545 | 0.159 |
| 19.418 | 4.5672 | 31.6 | 115 | 6637 | 0.166 |
| 19.943 | 4.44853 | 9.3 | 33.8 | 1966 | 0.181 |
| 21.31 | 4.1658 | 41.4 | 151 | 8714 | 0.192 |
| 22.797 | 3.89704 | 9.1 | 33.2 | 1921 | 0.201 |
| 23.587 | 3.76939 | 14.7 | 53.1 | 3082 | 0.171 |
| 24.345 | 3.65381 | 19.6 | 71.2 | 4116 | 0.208 |
| 25.169 | 3.53603 | 2.3 | 8.44 | 479 | 0.185 |
| 25.895 | 3.43955 | 5.4 | 19.7 | 1129 | 0.152 |
| 26.221 | 3.39654 | 5.0 | 15.6 | 1061 | 0.146 |
| 26.689 | 3.33736 | 11.1 | 40 | 2329 | 0.192 |
| 27.249 | 3.27088 | 5.0 | 19.1 | 1052 | 0.25 |
| 28.09 | 3.17445 | 5.6 | 20.2 | 1184 | 0.269 |
| 28.886 | 3.08881 | 3.2 | 11.4 | 666 | 0.219 |
| 30.129 | 2.96435 | 4.2 | 15.6 | 884 | 0.184 |
| 30.585 | 2.92187 | 1.8 | 6.17 | 369 | 0.313 |
| 31.413 | 2.84617 | 5.6 | 20.1 | 1174 | 0.212 |
| 32.162 | 2.78029 | 2.8 | 9.87 | 583 | 0.285 |
| 33.878 | 2.64293 | 1.1 | 2.36 | 236 | 0.109 |
| 34.419 | 2.60386 | 3.2 | 11.5 | 676 | 0.239 |
| 35.529 | 2.52408 | 6.0 | 21.9 | 1254 | 0.344 |
| 36.598 | 2.45267 | 3.0 | 11 | 621 | 0.269 |
| 37.924 | 2.37119 | 1.8 | 6.41 | 371 | 0.276 |
| 38.818 | 2.31643 | 1.4 | 2.74 | 295 | 0.172 |
| 39.398 | 2.28753 | 1.1 | 3.56 | 236 | 0.196 |

TABLE 2
Beta-lactam compounds

| No. | IUPAC Name | CAS No. |
|---|---|---|
| 1 | (2S,5R,6R)-6-[(R)-2-(4-ethyl-2,3-dioxo-1-piperazinecarboxamido)-2- | 61477-96-1 |
| | phenylacetamido]-3,3-dimethyl-7-oxo-4-thia-1-azabicyclo[3.2.0]heptane- | |
| | 2-carboxylic acid | |
| 2 | (2S,5R,6R)-3,3-dimethyl-7-oxo-6-(2-phenylacetamido)-4-thia-1- | 61-33-6 |
| | zabicyclo[3.2.0]heptane-2-carboxylic acid | |
| 3 | (5R,6S)-6-[(1R)-1-hydroxyethyl]-3-({2-[(iminomethyl)amino]ethyl}thio)- | 74431-23-5 |
| | 7-oxo-1-azabicyclo[3.2.0]hept-2-ene-2-carboxylic acid | |
| 4 | (5R,6S)-6-((R)-1-hydroxyethyl)-7-oxo-3-((R)-tetrahydrofuran-2-yl)-4-thia- | 106560-14-9 |
| | 1-azabicyclo[3.2.0]hept-2-ene-2-carboxylic acid | |
| 5 | (2S,5R,6R)-6-{[3-(2-chlorophenyl)-5-methyl-oxazole-4-carbonyl]amino}- | 61-72-3 |
| | 3,3-dimethyl-7-oxo-4-thia-1-azabicyclo[3.2.0]heptane-2-carboxylic acid | |
| 6 | (6R,7R,Z)-7-(2-(2-aminothiazol-4-yl)-2-(2-carboxypropan-2- | 72558-82-8 |
| | yloxyimino)acetamido)-8-oxo-3-(pyridinium-1-ylmethyl)-5-thia-1-aza- | |
| | bicyclo[4.2.0] oct-2-ene-2-carboxylate | |
| 7 | (6R,7R,Z)-3-(acetoxymethyl)-7-(2-(2-aminothiazol-4-yl)-2- | 63527-52-6 |
| | (methoxyimino)acetamido)-8-oxo-5-thia-1-azabicyclo[4.2.0]oct-2-ene-2- | |
| | carboxylic acid | |
| 8 | (6R,7R)-7-[(2Z)-2-ethoxyimino-2-[5-(phosphonoamino)-1,2,4-thiadiazol- | 400827-46-5 |
| | 3-yl]acetyl]amino]-3-[4-(1-methylpyridin-1-ium-4-yl)-1,3-thiazol-2- | |
| | yl]sulfanyl]-8-oxo-5-thia-1-azabicyclo[4.2.0]oct-2-ene-2-carboxylate | |
| 9 | (6R,7R,Z)-7-(2-(2-aminothiazol-4-yl)-2-(methoxyimino)acetamido)- | 88040-23-7 |
| | 3-((1-methylpyrrolidinium-1-yl)methyl)-8-oxo-5-thia-1-aza- | |
| | bicyclo[4.2.0]oct-2-ene-2-carboxylate | |
| 10 | (6R,7R)-3-{[(aminocarbonyl)oxy]methyl}-7-{[(2Z)-2-(2-furyl)-2- | 55268-75-2 |
| | (methoxyimino) acetyl]amino}-8-oxo-5-thia-1-azabicyclo[4.2.0]oct-2-ene- | |
| | 2-carboxylic acid | |
| 11 | (6R,7R)-7-{[(2Z)-2-(2-amino-1,3-thiazol-4-yl)-2- | 73384-59-5 |
| | (methoxyimino)acetyl]amino}-3-{[(2-methyl-5,6-dioxo-1,2,5,6-tetrahydro- | |
| | 1,2,4-triazin-3-yl)thio]methyl}-8-oxo-5-thia-1-azabicyclo[4.2.0]oct-2-ene- | |
| | 2-carboxylic acid | |
| 12 | (2S,5R,6R)-6-{[(2R)-2-amino-2-(4-hydroxyphenyl)-acetyl]amino}-3,3- | 26787-78-0 |
| | dimethyl-7-oxo-4-thia-1-azabicyclo[3.2.0]heptane-2-carboxylic acid | |
| 13 | 3-[5-(dimethylcarbamoyl) pyrrolidin-2-yl] sulfanyl-6-(1-hydroxyethyl)-4- | 119478-56-7 |
| | methyl-7-oxo-1-azabicyclo[3.2.0]hept-2-ene-2-carboxylic acid | |
| 14 | (6R,7R)-3-[(5-amino-4-{[(2-aminoethyl)carbamoyl]amino}-1-methyl-1H- | 689293-68-3 |
| | pyrazol-2-ium-2-yl)methyl]-7-({(2Z)-2-(5-amino-1,2,4-thiadiazol-3-yl)-2- | |
| | [(1-carboxy-1-methylethoxy)imino]acetyl}amino)-8-oxo-5-thia-1- | |
| | azabicyclo[4.2.0]oct-2-ene-2-carboxylate | |
| 15 | 5-amino-4-{[(2-aminoethyl)carbamoyl]amino}-2-{[(6R,7R)-7-({(2Z)-2- | 936111-69-2 |
| | (5-amino-1,2,4-thiadiazol-3-yl)-2-[(1-carboxy-1- | |
| | methylethoxy)imino]acetyl}amino)-2-carboxy-8-oxo-5-thia-1- | |
| | azabicyclo[4.2.0]oct-2-en-3-yl]methyl}-1-methyl-1H-pyrazolium | |
| | monosulfate | |

TABLE 3
Formulations of ceftolozane sulfate

| | Formulation A | Formulation B | Formulation C | Formulation D |
|---|---|---|---|---|
| Component | grams (mmol) | grams (mmol) | grams (mmol) | grams (mmol) |
| ceftolozane sulfate | 1.00* (1.5) | 1.00* (1.5) | 1.00* (1.5) | 0.47 (0.70) |
| L-arginine | 0.62 (3.56) | 0.93 (5.34) | 0.62 (3.56) | 0.29 (1.67) |
| Citric acid | 0.022 | 0.022 | 0.022 | 0.01 |
| NaCl | 0.49 | 0.49 | 0.49 | 0.23 |
| Tazobactam acid | — | 0.50 (1.67) | — | — |
| Polymorph Ia | — | — | 0.79 (1.67) | — |
| Sodium tazobactam | — | — | — | 0.21* (0.65) |
| pH | 5.81 | 6.72 | 6.30 | 5.89 |

*active weight

TABLE 4
Stability data for formulations of Table 1 at 25° C. (60% RH), T1 (1 month), T2 (3 month)

| | Formulation A | | | Formulation B | | | Formulation C | | | Formulation D | | |
|---|---|---|---|---|---|---|---|---|---|---|---|---|
| HPLC Peaks | T0 | T1 | T2 | T0 | T1 | T2 | T0 | T1 | T2 | T0 | T1 | T2 |
| Ceftolozane | 98.46% | 97.89% | 97.46% | 98.01% | 97.09% | 93.76% | 98.31% | 98.03% | 97.01% | 98.53% | 97.78% | 88.28% |
| Peak1(RRT | 0.29% | 0.42% | 0.80% | 0.39% | 0.85% | 2.57% | 0.30% | 0.46% | 0.83% | 0.21% | 0.45% | 6.08% |
| 0.150) | | | | | | | | | | | | |
| Peak3 (RRT | 0.09% | 0.06% | 0.14% | 0.10% | 0.11% | 0.59% | 0.09% | 0.05% | 0.18% | 0.06% | 0.05% | 2.03% |
| 0.429) | | | | | | | | | | | | |
| Peak4 (RRT | 0.05% | 0.08% | 0.09% | 0.06% | <0.03% | 0.13% | 0.06% | <0.03% | 0.08% | <0.03% | <0.03% | <0.03% |
| 0.612) | | | | | | | | | | | | |
| Peak5 (RRT | 0.11% | 0.12% | 0.12% | 0.12% | 0.12% | <0.03% | 0.11% | 0.12% | 0.12% | 0.11% | 0.11% | <0.03% |
| 0.872) | | | | | | | | | | | | |
| Peak7 (RRT | 0.89% | 0.90% | 0.96% | 0.88% | 0.95% | <0.03% | 0.89% | 0.85% | <0.03% | 0.88% | 0.92% | <0.03% |
| 1.262) | | | | | | | | | | | | |
| Peak8 (RRT | <0.03% | <0.03% | <0.03% | 0.10% | <0.03% | <0.03% | 0.07% | <0.03% | <0.03% | <0.03% | <0.03% | <0.03% |
| 1.394) | | | | | | | | | | | | |
| Peak9 (RRT | 0.04% | 0.04% | <0.03% | 0.04% | <0.03% | <0.03% | <0.03% | <0.03% | <0.03% | <0.03% | <0.03% | <0.03% |
| 1.684) | | | | | | | | | | | | |
| Others (RRT | <0.03% | <0.03% | <0.03% | <0.03% | <0.03% | 0.15% | <0.03% | <0.03% | 0.04% | <0.03% | <0.03% | 0.42% |
| 0.120) | | | | | | | | | | | | |
| Others (RRT | <0.03% | <0.03% | <0.03% | <0.03% | <0.03% | 0.10% | <0.03% | <0.03% | <0.03% | <0.03% | <0.03% | 0.28% |
| 0.653) | | | | | | | | | | | | |
| Others (RRT | <0.03% | <0.03% | <0.03% | <0.03% | <0.03% | 0.12% | <0.03% | <0.03% | <0.03% | <0.03% | <0.03% | 0.68% |
| 0.904) | | | | | | | | | | | | |
| Others (RRT | <0.03% | <0.03% | <0.03% | 0.05% | 0.38% | <0.03% | <0.03% | <0.03% | <0.03% | <0.03% | 0.17% | <0.03% |
| 1.22) | | | | | | | | | | | | |
| Others (RRT | <0.03% | <0.03% | <0.03% | <0.03% | <0.03% | 1.59% | <0.03% | <0.03% | 1.18% | <0.03% | <0.03% | 1.82% |
| 1.255) | | | | | | | | | | | | |

1-48. (canceled)

49. A composition obtained by a process comprising the steps of:
a) combining tazobactam acid, ceftolozane sulfate, sodium chloride, and L-arginine in an aqueous solution at a pH of 5-7; and
b) lyophilizing the aqueous solution to obtain the composition.

50. The composition of claim 49, wherein the tazobactam acid and ceftolozane sulfate are combined in the aqueous solution at a weight ratio of about 1 g ceftolozane active to 0.50 g tazobactam acid.

51. The composition of claim 49, wherein the aqueous solution further comprises citric acid.

52. The composition of claim 49, wherein the aqueous solution comprises an amount of L-arginine to provide a pH of about 6-7.

53. The composition of claim 49, wherein the composition is obtained by a process further comprising the step of filtering the aqueous solution through a 0.2 micron membrane and then lyophilizing the filtered composition.

54. The composition of claim 49, wherein the aqueous solution consists essentially of water, tazobactam acid, ceftolozane sulfate, sodium chloride, L-arginine and citric acid.

55. The composition of claim 49, wherein the lyophilized composition comprises ceftolozane active and tazobactam in a 2:1 weight ratio.

56. The composition of claim 49, wherein the aqueous solution comprises ceftolozane active and tazobactam in a 2:1 weight ratio.

57. The composition of claim 56, wherein the aqueous solution comprises an amount of L-arginine to provide a pH of about 6-7.

58. The composition of claim 57, wherein the aqueous solution further comprises a chelating effective amount of citric acid.

59. The composition of claim 58, wherein the composition is obtained by a process further comprising the step of filtering the aqueous solution through a 0.2 micron membrane and then lyophilizing the filtered composition.

60. A pharmaceutical composition comprising ceftolozane active and tazobactam in a 2:1 weight ratio, the composition obtained by a process comprising the steps of:
a) combining tazobactam acid, ceftolozane sulfate, sodium chloride, and L-arginine in an aqueous solution at a pH of 5-7; and
b) lyophilizing the aqueous solution to obtain a lyophilized composition; and
c) preparing the lyophilized composition as a pharmaceutical composition for intravenous administration.

61. The pharmaceutical composition of claim 60 prepared in a unit dosage form comprising about 1.0 g of ceftolozane active.

62. The composition of claim 60, wherein the tazobactam acid and ceftolozane sulfate are combined in the aqueous solution at a weight ratio of about 1 g ceftolozane active to 0.50 g tazobactam acid.

63. The composition of claim 60, wherein the aqueous solution further comprises citric acid.

64. The composition of claim 49, wherein the aqueous solution comprises an amount of L-arginine to provide a pH of about 6-7.

65. The composition of claim 49, wherein the composition is obtained by a process further comprising the step of filtering the aqueous solution through a 0.2 micron membrane and then lyophilizing the filtered composition.

66. The composition of claim 49, wherein the aqueous solution consists essentially of water, tazobactam acid, ceftolozane sulfate, sodium chloride, L-arginine and citric acid.

67. The composition of claim 49, wherein:
a) the aqueous solution comprises ceftolozane active and tazobactam in a 2:1 weight ratio; and
b) the aqueous solution comprises an amount of L-arginine to provide a pH of about 6-7.

68. A unit dosage form of a pharmaceutical composition for intravenous administration for the treatment of infections selected from the group consisting of: complicated intra-abdominal infections, complicated urinary tract infections and pneumonia, the unit dosage form comprising ceftolozane active and tazobactam in a 2:1 weight ratio, the ceftolozane active being present in the form of ceftolozane sulfate, wherein the pharmaceutical composition is obtained by a process comprising the steps of:
a) combining tazobactam acid, ceftolozane sulfate, sodium chloride, and L-arginine in an aqueous solution at a pH of 5-7, the aqueous solution comprising the ceftolozane active and tazobactam in a 2:1 weight ratio; and
b) lyophilizing the aqueous solution to obtain a lyophilized composition; and
c) preparing the lyophilized composition as a unit dosage form containing the pharmaceutical composition for intravenous administration.

69. The unit dosage form of claim 68, containing 1 g of ceftolozane active and about 0.5 g tazobactam for the treatment of infections selected from the group consisting of: complicated intra-abdominal infections and complicated urinary tract infections.

70. The unit dosage form of claim 68, wherein the aqueous solution contains an amount of sodium chloride in the range of 0.1-1.0 M.
